(19) United States
(12) Patent Application Publication
(10) Pub. No.: US 2024/0034936 A1
(43) Pub. Date: Feb. 1, 2024

(54) COMBINED MICROWAVE PYROLYSIS AND PLASMA METHOD AND REACTOR FOR PRODUCING OLEFINS

(71) Applicant: MICROWAVE SOLUTIONS GMBH, Riehen (CH)

(72) Inventors: Annelie Stapela, Riehen (CH); Mathys Johannes Rossouw, Rayton Gauteng (ZA)

(21) Appl. No.: 18/551,905

(22) PCT Filed: Mar. 24, 2022
(86) PCT No.: PCT/EP2022/057745

§ 371 Date: Sep. 22, 2023

(30) Foreign Application Priority Data
Mar. 24, 2021 (CH) 00312/21

(51) Int. Cl. C10B 53/07; C10B 55/00; H05H 1/46; C07C 4/04

(52) U.S. Cl. CPC C10B 53/07; C10B 55/00; H05H 1/461; C07C 4/04; H05H 2245/80

(57) ABSTRACT

The invention relates to a pyrolysis method for recovering at least one component from a feedstock material using a thermal treatment. The feedstock material is delivered to a pyrolytic chamber (1), exposed to a controlled atmosphere, and heated to a treatment temperature of the at least one component in the pyrolytic chamber (1) by applying microwave energy. The pyrolysis breakdown products are separated by fractional condensation and a targeted component is decomposed in microwave plasma. The microwave plasma is generated such that plasma temperature is varied over a temperature range including a decomposition and/or cracking temperature of the at least one component.

The invention relates to a combined microwave pyrolysis and plasma process preferably with a pulsed microwave plasma for extracting or recovering compounds of commercially valuable pyrolitical oils, hydrocarbons, monomers and chemicals (including plasticizers) as well as the cracking of feedstock (mentioned below) for the production of Olefins such as Ethylene and Propylene from plastics, mixed plastics, tires, rubber products, polymer composites, naphtha oils, ethane gas and bio oils as feedstock, using microwave energy. As part of the process some or all of the recovered compounds are treated in a microwave plasma to crack the polymers to shorter chain polymers, particularly ethane and propane

Feedstock materials such as tires, plastics, rubber products and polymer composites, which are used in a broad variety of products, constructions and manufacturing processes, represent a source of energy and raw material at the end of life of the products and constructions. Also, scrap materials accruing from manufacturing and production processes using such materials represent sources of energy and chemical building blocks. To support a circular economy these chemical building blocks should be recovered and used in chemical synthesis and/or manufacturing of products

The chemical industry greatly contributes to the Green House Gas emissions (GHG's). The decarbonization of chemical industries can contribute greatly to GHG's reduction. In the chemical industry, significant CO₂ emissions result from ethylene production, being the second most polluting high-volume commodity chemical after ammonia and accounting for ˜10% of the chemical industry GHGs. The high GHGs associated with existing ethylene production processes necessitates risk mitigating strategies such as carbon capture and storage, use of bio-based feedstock and materials, increase in the recycling of plastics, and shifting to renewable energy.

Different Microwave pyrolysis processes for rubber-or plastic waste exist as can be seen in the patents below. However these processes are different from the current invention. For example, efforts to recycle tires using microwave technology have been described in U.S. Pat. No. 5,507,927. Tires are fed into a microwave chamber as a tire waste stream and are exposed to a reduction atmosphere and microwave radiation. The temperature of the tires is monitored and a power input to the microwave generators is adjusted as required to obtain optimum temperature for reducing the tire material. The chamber is kept at slightly above atmospheric pressure to facilitate removal of gaseous products. Further, the reduction atmosphere is adjusted by increasing the concentration of reducing gases as the tire material breaks down. For reducing the tire material, twelve magnetrons are used, wherein each of them has 1.5 kW of power at a frequency of 2450 MHz.

Efforts to decompose plastics, which is not itself susceptible to microwave heating, have been described in U.S. Pat. No. 5,084,140. Plastics is mixed with carbonaceous material, such as waste tire material, and subjected to microwave radiation to heat the plastics to 400° C. to 800° C. and cause pyrolysis of the plastics.

Further, decomposition methods using plasma cracking for different hydrocarbons such as n-Hexadecane, lubricating oil, and heavy oil are described by Mohammad Reza Khani, Atieh Khosravi, Elham Dezhbangooy, Babak Mohammad Hosseini, and Babak Shokri. However, these methods have not been successfully applied to feedstocks and waste streams comprising tires, plastics, mixed plastics, rubber products, polymer composites, naphtha oils, ethane gas or bio oils.

In summary, the prior work has involved the use of single-frequency microwave radiation for recovering specific compounds from waste materials. However, known microwave systems have a low microwave energy penetration into a material to be treated, limiting the size of product that can be pyrolysed. Further, microwave energy at a frequency of 2.45 GHz is derived from electrical energy with a conversion efficiency of approximately only 50% for 2.45 GHz. The use of multiple small magnetrons in a pyrolysis reactor, that are typically shut on and off for temperature control, is inefficient and the temperature control is not very precise. Especially, pyrolytic oils, hydrocarbons, monomers and chemicals are very temperature sensitive resulting in yield and quality of the recovered compounds being affected negatively.

It is an object of the invention to provide a pyrolysis process and a pyrolysis reactor, combined with a plasma process and plasma reactor that improve the yield and quality of compounds recovered from feedstock such as tires, plastics, mixed plastics, rubber products, polymer composites, naphtha oils, ethane gas and bio oils, that allow for high volumes of feedstock to be processed, and that enhance economic and commercial viability of compounds recovered from these feedstock, especially for recovered olefins.

These and other objects, which will appear from the description below, are achieved by a pyrolysis and plasma decomposition method and reactor for recovering at least one component from a feedstock material using thermal and plasma decomposition as set forth in the appended independent claims. Preferred embodiments are defined in dependent claims.

According to the present invention the feedstock material is treated by the pyrolysis and plasma decomposition method for recovering at least one component. The method uses a thermal treatment, wherein the feedstock material is delivered to a pyrolytic chamber, exposed to a controlled atmosphere, and heated to a treatment temperature in the pyrolytic chamber by microwave energy to breakdown the feedstock material into pyrolysis breakdown products. The pyrolysis breakdown products are exposed to a microwave plasma, which is generated such that it generates a decomposition and/or cracking temperature of the at least one component to recover the component from the breakdown products.

According to a first variant method of the present invention the feedstock material is treated by the pyrolysis method by delivering the material to a pyrolytic chamber. In the chamber the feedstock material is exposed to a controlled atmosphere and to a thermal treatment to recover at least one component of the feedstock material.

Heating is accomplished by microwaves that directly and volumetrically heat the feedstock. The heating varies the temperature in the pyrolytic chamber over a temperature range including a cracking and/or decomposition temperature of the at least one component. Particularly the temperature in the pyrolytic chamber can be increased sequentially in successive heating steps or zones for applying different cracking and decomposition temperatures to the feedstock material for recovering differing components.

The pyrolysis reactor for recovering at least one component from the feedstock material according to the present invention comprises a pyrolytic chamber for accommodating the feedstock material and at least one microwave generator as a heat source for heating the feedstock material to a decomposition and/or cracking temperature of the feedstock material. Further, a control unit is provided, which comprises a microwave radiation control for generating microwave power using microwave frequencies between 300 MHz and 40000 MHZ, and a temperature control for controlling the treatment temperature for heating the feedstock material.

Preferably, the temperature control controls the temperature such that it sequentially varies or increases in the pyrolytic chamber. Advantageously, the temperature in the pyrolytic chamber remains below 1200° C., preferably below 1000° C., for recovering feedstock material as defined below.

The pyrolysis method and the pyrolysis reactor of the present invention are particularly suitable to recover components from carbon-based feedstock materials. The pyrolysis method is especially advantageous for recovering components from a feedstock or waste material stream comprising plastics, mixed plastics, rubber products, polymer composites, naphtha oils, ethane gas, bio-oils and/or tires. Plastics comprises ethylene (co)polymer, propylene (co)polymer, styrene (co)polymer, butadiene (co)polymer, polyvinyl chloride, polyvinyl acetate, polycarbonate, polyethylene terephthalate, (meth)acrylic (co)polymer, or a mixture thereof. Rubber products and tires comprise of natural and synthetic rubbers such as styrene butadiene rubber and butyl rubber. Naphtha oils comprise of a petroleum distillate, usually an intermediate product between gasoline and benzine, used for example as a solvent or fuel. Bio-oil is a liquid biofuel/oil produced from biomass.

In one embodiment, volatile components extracted from the pyrolysis chamber are passed through a fractional condensation system to condense out heavy components. The remaining volatiles containing the targeted components are then passed through a microwave plasma cracking step to further decompose the targeted components to for example olefins including ethane and propane.

Selected hydrocarbon materials, or combinations thereof, formed during the pyrolysis step can be separated from the other components by the fractional condensation system and then introduced to the plasma chamber, together with a carrying gas. The plasma decomposes the hydrocarbons to targeted smaller molecules that are in turn the feedstock for the production of new polymers.

In contrast to the state of the art the steps of the pyrolysis and plasma decomposition method according to the invention are applied in a combined process where the pyrolysis process is set up to provide specific components to the plasma process and the two processes are matched to one another.

An important aspect of this invention is that the pyrolysis conditions (e.g. microwave power, temperature and pressure), condenser temperature and plasma conditions are jointly optimised to achieve a high yield of the targeted component.

In the plasma chamber the targeted components are cracked by a non-equilibrium, low temperature pulsed microwave plasma. Gas flow rate, amount of carrying gas, plasma pressure and microwave frequency and microwave power input are selected to optimise the formation of for example olefins, which are a valuable product of the method. Preferably, the microwave plasma is generated by pulsed microwave radiation at frequencies between 300 MHz and 40000 MHz, with the frequency optimised for the component to be decomposed or cracked.

The microwave plasma is generated by the microwave plasma generator which comprises a power supply unit and a microwave source such as a solid-state microwave generator or electron tube (e.g. magnetron; triode; klystron or the like).

For plasma ignition the pyrolysis reactor may comprise an active impedance matching circuit. The active impedance matching circuit may be fitted between the microwave plasma generator and the plasma chamber. This arrangement maximises the electromagnetic field in the chamber during plasma initiation and then, once the plasma reaches steady state, to ensure maximum microwave power transfer into the plasma during steady state plasma operation. Typically, a Tesla coil or spark gap can be used to initiate the plasma.

The microwave plasma generator is advantageously operated with pulsed microwave radiation. Preferably, pulse widths of the radiation are in the range of 10 μs to 10 ms and duty cycles range from 1% to 50%. The radiation properties are selected according to the characteristics of the components to be decomposed by the plasma.

Preferably, the microwave generator(s) provide a continuously changeable heating energy inside the pyrolytic chamber. Thus, the temperature in the pyrolytic chamber is not simply altered in discrete or incremental steps, for example by switching on and off magnetrons as known from the prior art. The applied microwave power and chamber temperature can be adjusted in a precise manner over the range of decomposition temperatures required for recovering components of the feedstock material.

Preferably, for the plasma decomposition step the pulsed microwave generator provides accurate control of microwave power level, pulse width and pulse shape, to crack the reagent gases to valuable products.

In general, in the electromagnetic spectrum, microwaves lie between infrared and radio frequencies. The wavelengths of microwaves are between 1 mm and 1 m with corresponding frequencies between 300 GHz and 300 MHz, respectively. The two most commonly used microwave frequencies are 915 MHz and 2.45 GHz. Microwave energy is derived from electrical energy with a conversion efficiency of for example approximately 85% for 915 MHz but only 50% for 2.45 GHz. Most of the domestic microwave ovens use the frequency of 2.45 GHz. Compared with 2.45 GHz, the use of low frequency microwaves of 915 MHz can provide a substantially larger penetration depth which is an important parameter in the design of microwave cavity size, process scale up, and investigation of microwave absorption capacity of materials.

Further, the utilization of multiple small magnetrons for generating microwave radiation that are shut on and off for temperature control as known from the prior art are less efficient than a pulsed variable, high power microwave source as used in the pyrolysis method of the present invention. Heating from a pulsed variable, high power microwave source allows for very good temperature control during the recovery of components from the feedstock material. Most of the pyrolytic oils, hydrocarbons, monomers and chemicals, including plasticizers, are very temperature sensitive resulting in yield and quality being affected negatively in the absence of good temperature control as it can be provided by the variable, high power microwave source(s) of the invention.

The pyrolysis and plasma cracking method of the present inventions is especially useful for recovering an oil, a hydrocarbon, a monomer and/or a chemical plasticizer from a feedstock material. These components are extracted from the material by applying microwave heating in various zones of the microwave pyrolysis reactor and the zones operate independently from each other. Microwave radiation used is in the range of 300 MHz to about 40 GHz. The applied heating energy can be selected according to the decomposition or cracking temperature of a target recovery component for each of the zones. The energy can be changed variably between different decomposition temperatures of differing target recovery components. Thus, conditions in the chamber can be adapted to varying decomposition reactions of differing target recovery components.

Preferably, in the plasma cracking step the microwave plasma is designed for cracking at least one component of the feedstock material. Particularly, the pyrolysis and plasma cracking method and equipment of the invention is used advantageously for the cracking of Polymer, Naphtha, Ethane gas and bio oils feedstock, to produce olefins such as ethylene and propylene.

In one example the pyrolysis method is advantageously used for recovering at least one of an oil, a hydrocarbon, a monomer and/or a chemical plasticizer. Particularly, the method is used for recovering ethylene, propylene, methane, hydrogen, DL Limonene, isoprene, butadiene, benzene, toluene, o-xylene, m-xylene, p-xylene styrene and/or phthalates.

In one variant of the pyrolysis method according to the present invention the feedstock material is tempered in the pyrolytic chamber to around −161.5° C. to recover methane, to around −103.7° C. to recover ethylene, to around −47.6° C. to recover propylene, to around −4° C. to recover butadiene, to around 35° C. to recover isoprene, to around 80.1° C. to recover benzene, 110.6° C. to recover toluene, to around 138.3° C. to recover p-xylene, to around 139.1° to recover m-xylene, to around 144.4° C. to recover o-xylene, to around 145.2° C. to recover styrene, to around 178° C. to recover DL Limonene and/or to 300° C.-410° C. to recover phthalates. The indication of the temperatures being around these values shall be understood in that the temperature may deviate slightly from that value but not significantly enough to alter the pursued recovery process of the respective component.

In a further variant of the pyrolysis method according to the present invention olefins, particularly ethylene and propylene, are produced by cracking feedstock material comprising polymer, naphtha, ethane gas and/or bio oils.

Pyrolytic oils and gasses are complex mixtures of different chemical components with a wide range of molecular weights and boiling points. It has been found that condensation fractions obtained by fractional condensation of pyrolytic oils and gasses, that are boiling between −253° C. and 600° C., contain commercially valuable chemicals.

According to one aspect of the pyrolysis method of the present invention a pyrolytic oil is subjected to a fractional condensation at temperatures ranging from −253° C. to 600° C. to recover at least on component thereof. Preferably, a component recovered from the pyrolytic oil is selected from the group consisting of paraffins, naphthenes, olefins and aromatics.

The fractional condensation process preferably comprises the steps of a fast extraction of volatiles for reducing volatile residence time in the pyrolytic chamber. Next, the volatile gasses are condensed into different fractional oil components. Optionally, the fractioned components are subjected to a further fractional condensation to isolate at least one commercially valuable chemical selected from the group consisting of paraffins, naphthenes, olefins and aromatics.

During the fractional condensation process of pyrolysis breakdown products, evaporation steps or condensation steps, including cryogenic cooling, can be implemented to isolate targeted molecules for plasma treatment.

Particularly interesting components identified in the above condensation fractions are as mentioned above: methane recovered around −161.5° C., ethylene recovered around −103.7° C., propylene recovered around −47.6° C., butadiene recovered around −4° C., isoprene recovered around 35° C., benzene recovered around 80.1° C., toluene recovered around 110.6° C., p-xylene recovered 138.3° C., m-xylene recovered around 139.1°, o-xylene recovered 144.4° C., styrene recovered 145.2° C., DL Limonene recovered 178° C. and phthalates recovered between 300° C. and 410° C.

These components can be used as solvents and petrochemical feedstock in the synthesis of various polymers enabling resource circularity. For example, styrene is mainly used in the production of plastics, rubber and resins. Xylene is particularly useful in the production of polyester fibers; it is also used as solvent and starting material in the production of benzoic and isophthalic acids. Toluene is also used for the production of benzoic acid. DL Limonene is mainly used as a flavoring agent in the chemical, food and fragrance industries.

Thus, by pyrolysing the material in the pyrolysis chamber under controlled atmosphere, carrying out the fractional condensation of the pyrolytic oils to recover a fraction boiling in the range of about −253° C. to about 600° C. and utilizing the microwave plasma application also with a controlled atmosphere, as defined by the present invention, to further modify the pyrolysis products, it is possible to recover the above commercially valuable chemicals.

The controlled atmosphere in the pyrolytic chamber is advantageously a negative pressure environment with a pressure below 10 kPa.

The controlled atmosphere in the plasma reactor is advantageously a negative pressure environment with a pressure below 1 kPa containing the target components from pyrolysis as well as an optional inert carrying gas such as nitrogen or argon.

Further, the controlled atmosphere in both the pyrolytic chamber or the plasma reactor can be realized as a reactive atmosphere to modify the component or products of components formed during decomposition. The controlled atmosphere is advantageously defined by at least one reactive gas, which may include hydrogen, steam, methane, benzene, or a mixture of reactive gases, such as for example contained in syngas. Advantageously, reactive gases, particularly syngas, formed during the pyrolysis method are partially recycled through the reactor to promote alternate reactions or increase the yield of target liquid or gas products. The controlled atmosphere in the pyrolytic chamber can be selected and adapted according to a target component to be recovered by the pyrolysis method. Similarly the controlled atmosphere in the plasma reactor can be selected and adapted according to the target component to be formed by the plasma reaction.

Hydrocarbon oils and gases are produced by the pyrolysis method. It is desirable to increase the value of these pyrolytic oils and gases by a plasma dissociation step, with a view to obtain commercially valuable chemicals that enable carbon circularity and lessen the demand for fossil fuels.

In one variant of the plasma step of the invention the microwave plasma generators includes solid state pulse shaping that allows the amplitude and shape of the microwave pulses to be accurately controlled, and in turn for the specific control of the plasma flux and temperature. Active impedance matching circuits can ensure reliable plasma ignition and efficient power transfer during operation.

The thermal treatment of the feedstock material pursued by the microwave plasma application, particularly the microwave plasma application, results in the reaction of carbonaceous solids with high temperatures which leads to the production of gas and solid products. In the highly reactive microwave plasma zones there is a large amount of electrons, ions and excited molecules. Simultaneously, there is a high energy radiation, which rapidly heats any carbonaceous compounds in the feedstock. Volatile compounds are released and cracked resulting in recovery of hydrogen and hydrocarbons.

In a variation on the process described above the polymer material may be introduced to the pulsed microwave plasma directly, in the solid, liquid or gas phase and directly decomposed to the target compounds. The process controller regulates the microwave power input to control the plasma temperature and the products formed.

In summary, it is possible to pyrolyze, crack, extract and/or recover the above commercially valuable chemicals by utilizing the method based on a microwave plasma and pyrolysis system for the pyrolysis and recovery of pyrolytic oils, hydrocarbons, monomers and chemicals (including plasticizers) as well as for the cracking of feedstock for the production of olefins such as ethylene and propylene under a controlled atmosphere, and by optionally carrying out the fractional condensation of the pyrolytic oils or gasses to recover a fraction boiling in the range of about −253° C. to about 600° C. The yield and composition of the different chemicals depend on the feedstock.

Preferred embodiments of the invention will be described in the accompanying drawings, which may explain the principles of the invention but shall not limit the scope of the invention. The drawings illustrate:

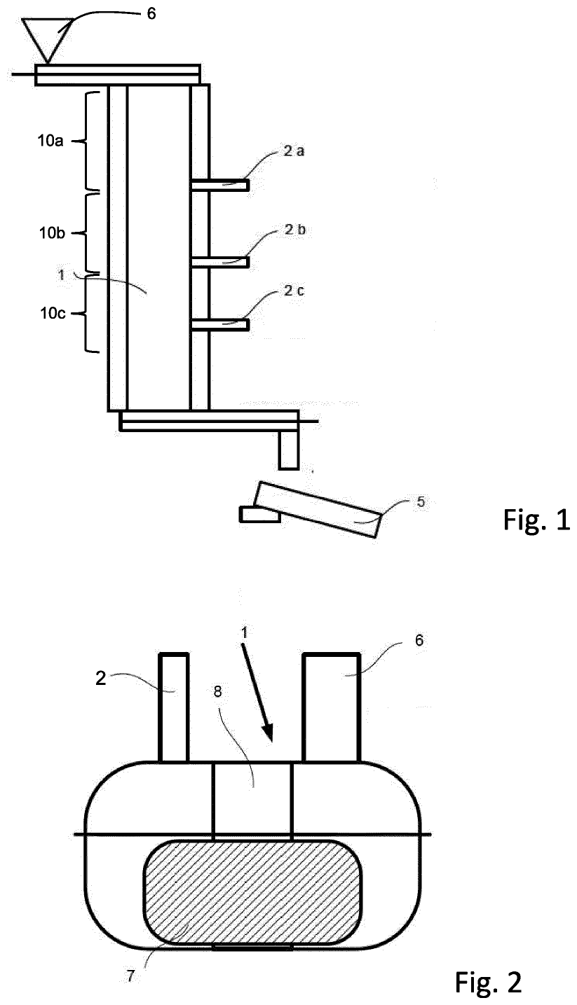

FIG. 1: a schematic diagram of a first example set up of a pyrolysis reactor according to the invention, and

FIG. 2: a schematic view of a second example set up describing a pyrolytic chamber of a pyrolysis reactor according to the invention.

In the following, two example embodiments of a pyrolysis reactor according to the present invention are described which are suitable to perform a pyrolysis method for recovering at least one component from a feedstock material using a thermal treatment according to the invention. In both of the embodiments, the pyrolysis reactor for thermal decomposition and/or cracking of feedstock materials, particularly pyrolytic oils, hydrocarbons, monomers and chemicals from feedstock and waste streams such as tires, plastics, mixed plastics, rubber products polymer composites, naphtha oils, ethane gas and bio oils, comprises a pyrolytic chamber 1 for accommodating the feedstock material. Further, the example embodiments of the pyrolysis reactor comprise at least one microwave generator having a microwave radiation source as a heat source for heating the feedstock material to a decomposition and/or cracking temperature of the feedstock material.

A process control unit, such as a programmable logic controller (PLC), is used to control the pyrolysis process according to the invention. Advantageously, the temperature control operates the microwave generator to sequentially vary or increase the temperature in the pyrolytic chamber 1. The control unit also comprises a microwave radiation control for generating a microwave plasma using microwave frequencies between 300 MHz and 40000 MHZ to the feedstock material, and a temperature control for controlling the decomposition and/or cracking temperature of the feedstock material inside the plasma reactor.

The two example embodiments mainly differ in the design of their pyrolytic chamber, while other features of the reactor and steps of the method are the same. Therefore, structural features of the reactor and explanations of method steps which are suitable for both example embodiments shall be regarded as interchangeable between the two example embodiments.

For example, for both example embodiments it is advantageous to define that the temperature range of the pyrolysis method extends between ambient and 1200° C., particularly between ambient and 1000° C. The example embodiments are suitable to pyrolyse a pyrolytic oil and subjecting it to a fractional condensation at a temperature range between −253° C. and 600° C. The pyrolytic chamber may comprise a controlled atmosphere in form of a negative pressure environment, particularly a pressure below 10 kPa, or the controlled atmosphere is defined by at least one reactive gas, particularly a gas selected from hydrogen, steam, carbon monoxide, methane, benzene or a mixture thereof. The example embodiments allow for the extraction of volatile gasses from the pyrolytic chamber and condensing the gasses into different fractional oils. In the same way other features and steps apply to both of the embodiments.

FIG. 1 shows an example embodiment of the pyrolytic reactor in the form of a continuous flow retort with an elongated design. For example, it may comprise a conveyor to deliver feedstock material to the pyrolytic chamber 1 and transfer the material through the chamber while components thereof are decomposed.

For example, complete tyres, plastics, rubber products and polymer composites can intermittently be fed into the pyrolytic chamber 1 through a feed port 6 at a first end of the chamber. An air lock system with means for purging of oxygen can be provided at the first end as well.

Pyrolysis gases are drawn off at intervals along the length of the pyrolytic chamber 1, wherein successive exit ports 2 are provided at zones of increasing chamber temperature and different gases or compounds can be collected though the exit ports. In the variant of FIG. 1, gases are collected from exit ports 2a, 2b and 2c at three positions located along the length of the chamber, which ports correspond to three different recovery components. Solid products may be discharged through an airlock system at an end of the pyrolytic chamber 1 and may be separated using a suitable method, such as a vibrating screen 5 or the like.

The control unit can regulate the microwave power input to the pyrolytic chamber and control the temperature of the feedstock material at various successive heat zones 10a, 10b and 10c along the pyrolytic chamber 1 in a sequentially increasing treatment temperature fashion. Also, the control unit comprises a microwave radiation control for generating a microwave plasma of variable energy at frequencies between 300 MHz and 40000 MHz inside the plasma reactor.

In the example pyrolysis reactor shown in FIG. 1 feedstock material is introduced into the feed port 6 at a first end of the pyrolytic chamber 1 by a conveyor and transported along the length of the pyrolytic chamber 1. In the course of sequentially increasing treatment temperatures the pyrolytic chamber and the feedstock material respectively are first heated to a first decomposition temperature of a first component of the feedstock material within a first heat zone by microwave heating. First products may be evacuated through a first exit port 2a.

The pyrolytic chamber 1 can be designed as a continuous reactor and the subsequent heat zones can merge into each other.

At a second end of the pyrolytic chamber 1 further recovery components or feedstock remnants may be discharged through the airlock system.

FIG. 2 shows a schematic view of a pyrolytic chamber 1 of a second example embodiment of the pyrolysis reactor according to the present invention. The reactor has the form of a batch reactor such as a pressure vessel that opens to accept a load of feedstock material such as rubber tyres. For example, the pyrolytic chamber 1 of the reactor is of circular shape and may be opened at the top of the circular chamber.

In the shown example embodiment the reactor is loaded with a single tyre 7. microwave plasma is applied to the pyrolytic chamber 1 through feed ports 6 in a roof of the chamber. Electrical elements or burning off of some of the pyrolysis products may provide heating of the chamber walls to assist with heating and to prevent condensation inside the vessel. The pulsed microwave plasma is introduced through a number of microwave feed ports 6 on the roof of the vessel that are arranged in positions and orientations that ensure a uniform distribution of microwave radiation in the chamber 1. The chamber may also be in the shape of an annulus where the central portion 8 is removed to reduce unoccupied volume in the pyrolytic chamber 1.

In the batch reactor the temperature of the feedstock material can be increased in heating steps to the decomposition or cracking temperature of differing components to be recovered. Condensate can be collected in a storage dedicated to that component, while switching between condensate storages for each step of the sequential pyrolysis process. During the process the reactor wall temperature can also be increased in heating steps to prevent re-condensation of the volatiles in the reactor. The temperature can be controlled by the control unit. In each heating step recovery components are extracted from the pyrolytic chamber 1 through the exit port 2 and can enter a condenser system.

The PLC also monitors the temperature of the material, reaction vessel and volatiles exiting the reactor at the gas exit ports 2, and at the various decomposition heat zones 10a, 10b and 10c along the length of the reactor. Online and offline analysis of the pyrolysis products may also be used to provide inputs to the control unit. Based on the data collected the process control unit regulates the microwave power input into the heat zones and the residence and travelling time of the material in the reactor. By varying the microwave powering the different heat zones of the reactor the material is heated to predefined temperatures corresponding to decomposition or cracking temperatures of differing material components. This allows these components to decompose or crack in their corresponding heat zone and the volatiles produced during the treatment of that component can be collected in a dedicated condenser and collection vessel. In subsequent heat zones the remaining material components are for example heated to successively higher decomposition or cracking temperatures, each time extracting the volatile components associated with the different material components and collecting it in separate condenser systems. This sequential decomposition of differing material components allows the different components produced to be collected separately. The volatile components produced during each pyrolysis step are decomposed in the pulsed microwave plasma reactor and the decomposition products collected in storage vessels where it may be isolated by distillation.

It is also an objective of the process to bypass the condenser system and subject all the volatile components formed during pyrolysis, to the plasma decomposition.

The pyrolysis method and the pyrolysis reactor according to the present invention relies on the fact that each of the material components present in a feedstock material has different boiling points and microwave absorption properties. The application of pulsed microwave plasma using frequencies between 300 MHz and 40000 MHZ to sequentially increase the temperature in the pyrolytic chamber over a temperature range including the decomposition and/or cracking temperature of recovery components ensures a high yield of recovery and high quality of the recovered components. Also, a broad variety of components can be recovered due to the wide range of possible treatment temperatures.

### LIST OF REFERENCE NUMBERS

1 pyrolytic chamber
2a,b,c exit ports
5 vibrating screen
6 feed port
7 rubber tyre
8 centre portion
10a,b,c heat zones

It is claimed:

1. Pyrolysis and plasma decomposition method for recovering at least one component from a feedstock material using a thermal treatment, wherein the feedstock material is
delivered to a pyrolytic chamber,
exposed to a controlled atmosphere, and
heated to a treatment temperature in the pyrolytic chamber by microwave energy to breakdown the feedstock material into pyrolysis breakdown products,
and wherein pyrolysis breakdown products are exposed to a microwave plasma, which is generated such that it generates a decomposition and/or cracking temperature of the at least one component.

2. Pyrolysis and plasma decomposition method according to claim 1, wherein the microwave plasma is generated by a microwave radiation at frequencies between 300 MHz and 40000 MHZ.

3. Pyrolysis and plasma decomposition method according to claim 1, wherein the microwave plasma is generated by pulsed microwave radiation.

4. Pyrolysis and plasma decomposition method according to claim 1, wherein the temperature in the pyrolytic chamber remains below 1200° C.

5. Pyrolysis and plasma decomposition method according to claim 1, wherein the feedstock material is a feedstock or waste material stream comprising plastics, mixed plastics, rubber products, polymer composites, naphtha oils, ethane gas, bio oils and/or tires.

6. Pyrolysis and plasma decomposition method according to claim 1, wherein the at least one recovered component is an oil, a hydrocarbon, a monomer and/or a chemical plasticizer.

7. Pyrolysis and plasma decomposition method according to claim 1, wherein the at least one recovered component is ethylene, propylene, methane, hydrogen, DL Limonene, isoprene, butadiene, benzene, toluene, o-xylene, m-xylene, p-xylene styrene and/or phthalates.

8. Pyrolysis and plasma decomposition method according to claim 1, wherein the feedstock material is tempered to around −252.9° C. to recover hydrogen, to around −161.5° C. to recover methane, to around −103.7° C. to recover ethylene, to around −47.6° C. to recover propylene, to around −4° C. to recover butadiene, to around 35° C. to recover isoprene, to around 80.1° C. to recover benzene, 110.6° C. to recover toluene, to around 138.3° C. to recover p-xylene, to around 139.1° to recover m-xylene, to around 144.4° C. to recover o-xylene, to around 145.2° C. to recover styrene, to around 178° C. to recover DL Limonene and/or to 300° C.-410° C. to recover phthalates.

9. Pyrolysis and plasma decomposition method according to claim 1, wherein volatile components extracted from the pyrolysis chamber are passed through a fractional condensation system.

10. Pyrolysis and plasma decomposition method according to claim 1, wherein olefins, particularly ethylene and propylene are produced by cracking feedstock material comprising polymer, naphtha, ethane gas and/or bio oils.

11. Pyrolysis and plasma decomposition method according to claim 1, wherein the feedstock material comprises a pyrolytic oil or gas and the feedstock material is subjected to a fractional condensation at a temperature range between −253° C. and 600° C. resulting in at least one condensation fraction.

12. Pyrolysis and plasma decomposition method according claim 11, wherein the at least one condensation fraction is subjected to a further fractional condensation isolate paraffins, naphthenes, olefins and/or aromatics.

13. Pyrolysis and plasma decomposition method according to claim 1, wherein the controlled atmosphere is a negative pressure environment applied in the pyrolytic chamber, particularly a pressure below 10 kPa.

14. Pyrolysis and plasma decomposition method according to claim 1, wherein the controlled atmosphere is defined by at least one reactive gas, particularly a gas selected from hydrogen, steam, carbon monoxide, methane, benzene or a mixture thereof.

15. Pyrolysis and plasma decomposition method according to claim 1, wherein a temperature of the microwave plasma is controlled by varying an amplitude and shape of microwave radiation pulses that generate the microwave plasma.

16. Pyrolysis and plasma decomposition method according to claim 1, wherein a temperature and microwave power input varies in successive zones of the pyrolytic chamber.

17. Pyrolysis reactor for recovering at least one component from a feedstock material using thermal decomposition, comprising a pyrolytic chamber for accommodating the feedstock material and at least one microwave generator as a heat source for heating the feedstock material to a pyrolysis temperature of the feedstock material, as well as a plasma treatment chamber with microwave generator to produce a microwave plasma, with a control unit, which comprises a microwave radiation control for generating a microwave plasma using microwave frequencies between 300 MHz and 40000 MHZ, and a temperature control controlling a decomposition temperature of the feedstock material.

18. Pyrolysis reactor according to claim 17, which comprises an active impedance matching circuit for plasma ignition in the plasma chamber.
